(19) United States
(12) Patent Application Publication
(10) Pub. No.: US 2002/0177846 A1
(43) Pub. Date: Nov. 28, 2002

(54) Vaporous delivery of thermal energy to tissue sites

(76) Inventors: Peter M.J. Mulier, Stillwater, MN (US); Filip Mulier, Vadnais Heights, MN (US); Michael F. Hoey, Shoreview, MN (US)

Correspondence Address: SHUMAKER & SIEFFERT, P. A., 8425 SEASONS PARKWAY, SUITE 105, ST. PAUL, MN, 55125, US

(21) Appl. No.: 10/094,952

(22) Filed: Mar. 6, 2002

Related U.S. Application Data
(60) Provisional application No. 60/273,693, filed on Mar. 6, 2001.
(60) Provisional application No. 60/325,057, filed on Sep. 26, 2001.

(51) Int. Cl. A61B 18/04

(52) U.S. Cl. 606/27; 607/96; 607/100

(57) ABSTRACT

Many surgical applications require the heating of a tissue site. The heating of the site may be for the purpose ablating the tissue, shrinking the tissue, coagulating the tissue, cauterizing the tissue, or the like. Vapor provides a medium for transfer of thermal energy to a tissue site. The vapor rapidly heats the tissue rapidly upon release of thermal energy in the phase change of the vapor from gas to liquid. By changing attributes of the vapor such as duration, direction, pressure, volume, and temperature, the effect, i.e. ablation, coagulation, or the like, of the heating can be controlled.

This application claims priority from U.S. Provisional Application Serial No. 60/273,693, filed Mar. 6, 2001, and U.S. Provisional Application Serial No. 60/325,057, filed Sep. 26, 2001, the entire content of each of which is incorporated herein by reference.

### TECHNICAL FIELD

The invention relates to medical devices and, more particularly, to medical devices for treatment of tissue sites of a mammal or human patient.

### BACKGROUND

A variety of techniques are available for treatment of abnormal or diseased tissue sites. Abnormal or diseased tissue sites may be characterized by the presence of malignant or benign tumors, enlargement, bleeding or other undesirable conditions. Often, removal of abnormal or diseased tissue is desirable. For example, a tissue site may be physically resected using a scalpel or other cutting tool to remove tissue. Some techniques for treatment of abnormal or diseased tissue sites involve removal of tissue by electrosurgical, laser, or radio frequency (RF) ablation.

In general, existing ablation techniques generate thermal energy at the tissue site. The thermal energy heats the abnormal or diseased tissue, killing abnormal or diseased cells within the tissue. The thermal energy also may kill surrounding tissue that is not abnormal or diseased in order to ensure that the targeted tissue is eradicated. The ablation process generally results in vaporization of cells at the tissue site. The ablated tissue may be removed physically, e.g., by aspiration. Alternatively, the ablated tissue may be slowly eliminated by the normal function of the immune system.

Thermal energy also may be used to stop bleeding at a tissue site. In some cases, the bleeding may be caused by disease or an abnormal tissue condition. In other cases, the bleeding may be incident to a surgical procedure. Thermal energy may be applied, in either case, to cauterize an area of tissue to stop the bleeding. An electrosurgical probe, for example, may generate thermal energy within a tissue site by passing current between two or more electrodes in contact with the tissue.

### SUMMARY

In general, the invention is directed to vaporous delivery of thermal energy to a site associated with a patient, e.g., a tissue site. A vaporous medium, e.g., steam, may be used to store thermal energy for application to a tissue site. Upon application to the tissue site, the vaporous medium transfers the stored thermal energy to the tissue site. The thermal energy can be used to perform a variety of surgical procedures including tissue ablation, tissue shrinkage, coagulation, hemostasis, cauterization, and heat sealing of living tissue.

The vaporous medium can be directed with relative precision to effect ablation of a selected portion of a tissue site. For example, a variety of surgical devices, including handheld instruments, catheters, and the like, can be configured to emit controlled amounts of the vaporous medium to selected areas for limited durations. The vaporous medium may be generated externally and delivered to the surgical instrument, or generated internally within the surgical instrument.

Moreover, a surgical device configured to emit a vaporous medium may further integrate other surgical components, such as forceps, blades, snares, or the like. The integrated features may be constructed for conventional resection, electrosurgical ablation, radio frequency (RF) ablation, laser ablation, or the like. The surgical device may further carry components for aspiration, irrigation, illumination and imaging.

The vaporous medium may take the form of steam, i.e., water vapor, or gaseous forms of other substances. Advantageously, vaporous media such as steam tend to be hyperechoic, promoting ultrasonic imaging of the area near a treatment site. In addition, vaporous media have a very high energy storage capacity. For example, steam can transfer up to six times more heat energy than hot water and up to eight hundred times more energy than hot air.

The vaporous medium may be loaded with additional substances. In particular, in some embodiments, the vaporous medium may carry chemotherapeutic substances for eradicating diseased tissue cells, substances for preventing infection, substances for conditioning tissue cells to accelerate ablation or other procedures, contrast agents for promoting imaging of diseased tissue cells, and other useful ancillary substances. In addition, some substances may be added to modify the electrical conductivity or impedance of the vaporous medium.

In one embodiment, the invention is directed to a method in which a vaporous medium is generated. The vaporous medium is delivered to a tissue site of a patient via a surgical instrument. The vaporous medium transports thermal energy to the tissue site, which in turn heats the tissue site.

In another embodiment, the invention is directed to an apparatus used for generating and delivering a vaporous medium to a tissue site of a patient. The apparatus includes a liquid supply. The apparatus further includes a source of thermal energy. The thermal energy is used to heat and evaporate the liquid. The evaporation of the liquid creates a vaporous medium. The apparatus delivers the vaporous medium to a tissue site of a patient via a vapor outlet.

In yet another embodiment, the invention is directed to a system for generating a vaporous medium and delivering it to a tissue site. The system includes a source that generates the vaporous medium. The system further comprises a surgical instrument that delivers the vaporous medium to a tissue site of a patient.

In an added embodiment, the invention is directed to a device that generates a vaporous medium internally. The device includes an outer housing that houses the internal structures of the device, and an inner liquid conduit that receives liquid from a liquid supply conduit. The device also includes a chamber adjacent to the distal end of the device. The chamber is in fluid communication with an inner liquid conduit via an inlet port, and in fluid communication with a vapor outlet via an exit port. The device further comprises electrical leads that extend from the proximal end of the housing. One of the leads is coupled to the chamber wall and the other lead is coupled to the inner liquid conduit. A radio frequency generator is coupled to the leads at the proximal end of the device. The radio frequency generator creates a radio frequency generator to heat the liquid in the chamber. The liquid in the chamber evaporates, creating a vaporous medium. The vaporous medium is emitted from the chamber via the vapor outlet.

In another embodiment, the invention provides a method comprsing delivering a vaporous medium to a tissue site of a patient, the vaporous medium having a temperature selected to cause at least one of ablation, hemostasis and tissue shrinkage within a portion of the tissue site.

In a further embodiment, the invention provides a device comprising a housing to contain a vaporous medium, and a port to direct the vaporous medium at a tissue site of a patient.

In an added embodiment, the invention provides a method comprising delivering a vaporous medium to a tissue site of a patient, wherein a portion of the vaporous medium travels within interstitial spaces between tissue cells at the tissue site.

In various embodiments, the invention may provide one or more advantages. For example, a vaporous medium such as steam is generally hyperechoic, permitting visibility in ultrasound images. Accordingly, physicians may be able to monitor the application of vaporous media such as steam during a surgical procedure.

As another advantage, transfer of thermal energy using a vaporous medium does not generally require tissue contact, and can reduce physical disturbance of diseased tissue cells while they are still viable. The vaporous medium can be sprayed at the tissue site from a distance, and has a controllable range of effect. Consequently, when used for coagulation, tissue does not generally adhere to the delivery instrument.

In addition, the vaporous medium generally does not cause the unpleasant odor associated with other ablation and cauterization techniques, because there is no vaporization of tissue. Further, vaporous media such as steam are capable of transferring large amounts of thermal energy per unit volume of the emitted steam. Vaporous media are also capable of combination with other techniques such as electrosurgical cautery or ablation, laser ablation or radio frequency ablation.

A vaporous medium such as steam also may be electrically nonconductive, permitting combination of steam delivery with electrosurgical procedures such as RF ablation, hemostasis, and the like. In other embodiments, however, additional substances carried by the vaporous medium may impart some degree of electrical conductivity.

Further, the vaporous medium may provide penetration of thermal energy into interstitial channels between tissue cells. Hence, the vaporous medium not only contacts surface tissue, but also can penetrate deep into the tissue site, distributing thermal energy over a large cell surface area. In this manner, the vaporous medium may be able to exploit the microanatomy of tissue to increase the efficiency of heat transfer to the cells, in comparison to cell-to-cell conductance of heat.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an embodiment of a medical vapor delivery system.

FIG. 2 is a block diagram illustrating another embodiment of a medical vapor delivery system.

FIG. 3 is a schematic diagram illustrating an exemplary embodiment of a medical vapor delivery system employing a resistive heater to generate vapor.

FIG. 4 is a schematic diagram illustrating an exemplary embodiment of a medical vapor delivery system employing a radio frequency heater to generate vapor.

FIG. 5 is a schematic diagram illustrating an exemplary embodiment of a medical vapor delivery system comprising a vapor source internal to a surgical instrument.

FIG. 6 is a schematic diagram illustrating another embodiment of a medical vapor delivering system comprising a vapor source internal to a surgical instrument.

FIG. 7 is a cross-sectional side view illustrating a surgical tool that generates vapor internally.

FIG. 8 is a schematic diagram illustrating a vapor hemostasis wand.

FIG. 9 is a schematic diagram illustrating a vapor cutting hemostat.

FIG. 10 is a schematic diagram illustrating a vapor scalpel.

FIG. 11 is a schematic diagram illustrating a profile on view of vapor scalpel.

FIG. 12 is a schematic diagram illustrating a vapor coagulating scissors.

FIG. 13 is a schematic diagram illustrating an endoscopic vapor coagulating scissors.

FIG. 14 is a schematic diagram illustrating a vapor forceps.

FIG. 15 is a schematic diagram of a vapor scissor forceps.

FIG. 16 is a schematic diagram of an endoscopic vapor forceps.

FIG. 17 is an enlarged schematic diagram illustrating the jaws of the vapor forceps of FIG. 14-17.

FIG. 18 is a schematic diagram of a medical vapor delivery system capable of controlling the temperature and pressure of a vapor spray.

FIG. 19 is a schematic diagram illustrating a vapor catheter being used for intraluminal shrinking.

FIG. 20 is a schematic diagram illustrating an insulated vapor needle being used for tissue ablation.

FIGS. 21-23 are schematic diagrams of catheters for delivery of a vaporous medium.

### DETAILED DESCRIPTION

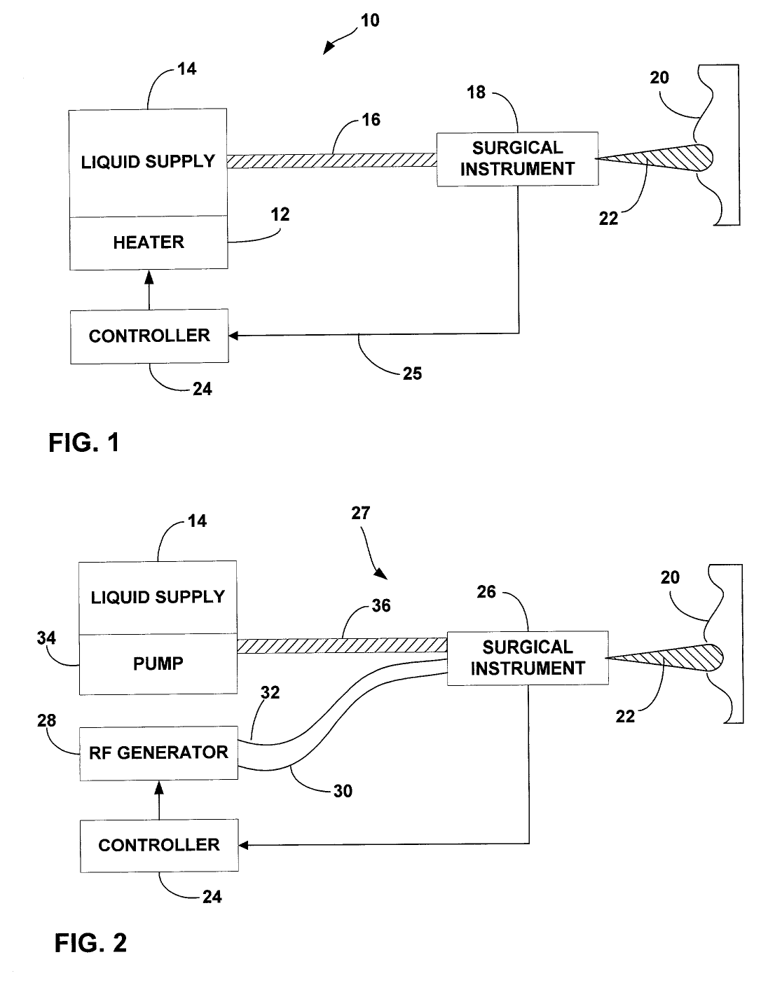

FIG. 1 is a block diagram illustrating an embodiment of a medical vapor delivery system 10. System 10 may deliver a vaporous medium to a site, e.g., a tissue site, internal or external to a patient, e.g., mammal or human. In the example of FIG. 1, system 10 may include a heater 12 that heats the contents of a liquid supply 14. Heater 12 heats liquid supply 14 so that the liquid contents evaporate. System 10 transports the resulting vapor through a vapor conduit 16 to a surgical instrument 18. As the liquid evaporates and the vapor expands, the pressure of the vapor within liquid supply 14 serves to transport the vapor through vapor conduit 16. Conduit 16 may be flexible to facilitate positioning of surgical instrument 18 relative to a tissue site 20. A pump may also be used, in some embodiments, to assist in transporting the vapor through vapor conduit 16 to surgical instrument 18. Heater 12 may be any of a variety of heating devices, such as a resistive heater, a radio frequency (RF) heater, a microwave heater, a laser, or a high intensity focused ultrasound generator.

Liquid supply 14 may contain a biocompatible liquid such as water, saline, Ringer's solution, or the like. The liquid supply may optionally contain one or more additional substances such as salt, iodine, lidocaine, or chemotherapeutic agents, e.g., to aid in the efficacy of particular surgical procedures. As an example, ablation of tissue may be accompanied by delivery of chemotherapeutic substances for treatment of surrounding tissue. As a further example, salt or other substances may be provided to draw fluid from cells within a tissue site to provide a dehydrating effect that accelerates the effect of an ablation process on tissue cells. Some of the additional substances may not be evaporated with the liquid supply, but can be made small enough to be carried by the vapor through vapor conduit 16, into surgical instrument 18 and to a desired tissue site 20. Vapor conduit 16 may be insulated to prevent a significant decrease in the temperature of the vapor as it travels along the vapor conduit. A significant decrease of the temperature of the vapor within conduit 16 may cause the vapor to condense and lose thermal energy.

Surgical instrument 18 may take the form of a handheld instrument such as a forceps, hemostasis wand, scalpel, or the like. Alternatively, surgical instrument 18 may be designed for laparoscopic or intraluminal deployment. In particular, in some embodiments, surgical instrument 18 may take the form of a catheter that is deployed intraluminally to the tissue site via a vessel within the patient. Surgical instrument 18 may deliver the vapor to a tissue site 20 in the form of a vapor spray 22.

Vapor spray 22 delivers thermal energy to tissue site 20. The thermal energy delivered by vapor spray 22 can be much larger than the amount of thermal energy that ordinarily would be delivered by a heated liquid. In particular, vapor spray 22 condenses at the tissue site, releasing a substantial amount of energy as a byproduct of the resulting phase change from gas to liquid. The thermal energy transported to tissue site 20 as a result of the phase change of vapor spray 22 heats the tissue site rapidly.

As shown in FIG. 1, vapor spray 22 may have a generally plume-like shape that can be directed to heat a relatively confined area near tissue site 20, thereby reducing undesired heating of adjacent tissues. In addition, vapor spray 22 may be pressurized to permit emission of the vapor spray at a desired trajectory. The vapor spray may penetrate interstitial channels between cells, and distribute thermal area over a large cell surface area. In this manner, the transfer of thermal energy by the vapor may be more effective than cell-to-cell conduction of heat energy, permitting procedures like ablation to be accomplished very quickly.

A controller 24 can be provided to control heater 12. Controller 24 may be responsive to a switch, button or other control input actuated by a surgeon to activate heater 12. The control input may be carried on surgical instrument 18, as represented by line 25, or on controller 24. When a surgeon actuates the control input, controller 24 activates heater 12 to increase the temperature within liquid supply 14. In addition, controller 24 may be configured to open or close a valve that permits flow of vapor from liquid supply 14 to conduit 16. Alternatively, a control valve may be provided between conduit 16 and surgical instrument 18 or at an output port of surgical instrument 18. In each case, the contents of liquid supply 14 may be pre-evaporated and pressurized for immediate availability when the surgeon is ready to use surgical instrument 18.

Surgical instrument 18 may further include one or more sensors to measure various operational parameters during a surgical procedure. Controller 24 may be configured to control heater 12 based on the measurements. For example, controller 24 may control heater 12 to achieve desired levels of temperature, pressure, or volume in vapor spray 22. In one embodiment, a thermocouple, thermistor or other temperature-sensing device may be incorporated in surgical instrument 18 to measure temperature proximate to target tissue site 20. Other devices for measuring temperature, including optical sensors or microbolometers, may be used.

When a particular temperature is reached at the tissue site for a sufficient amount of time, controller 24 may respond by adjusting the level of heat applied to liquid supply 14 by heater 12, deactivating the heater, or closing a valve to stop flow of vapor through conduit 16 or through surgical instrument 18. In another embodiment, a flow meter or the like may be incorporated in surgical instrument 18. The flow meter may be used to measure the flow, pressure, or volume of vapor spray 22 emitted from surgical instrument 18 to target tissue site 20. For example, if the pressure of vapor spray 22 is insufficient to produce a desired depth of ablation, controller 24 may adjust the level of heat applied to liquid supply 14 by heater 12 or adjust a pressure regulator that may be provided within liquid supply 14, vapor conduit 16 or surgical instrument 18. If the level of heat applied by heater 12 is increased, the contents of liquid supply 14 generally will evaporate more rapidly, causing an increase in pressure of the vapor.

In yet another embodiment, a sensor may be incorporated in surgical instrument 18 to detect the progress of the surgical procedure. For example, an optical sensor could detect the color of tissue site 20 as an indication of the progress of the procedure. Alternatively, a sensor could measure conductivity or other physiological parameters of the tissue at tissue site 20 to assess the progress of the surgical procedure. As an example, if tissue site 20 were a color indicating the desired result of the procedure, controller 24 could adjust the level of heat applied to liquid supply 14 by heater 12, deactivate heater 12, or adjust any of a variety of control valves or pressure regulators within system 10.

FIG. 2 is a block diagram illustrating an example of another embodiment of a medical vapor delivery system 27. In the example of FIG. 2, medical vapor delivery system 27 may incorporate a source of vapor that is internal to a surgical instrument 26. In particular, in the example of FIG. 2, medical vapor delivery system 27 includes a radio frequency (RF) generator 28 having a pair of leads 30, 32 coupled to surgical instrument 26. RF generator 28 supplies radio frequency current to surgical instrument 26 via leads 30, 32. RF generator 28 may be a standard RF generator that is commonly available in electrosurgical units used in operating rooms. As will be described, leads 30, 32 may be electrically coupled to terminals within surgical instrument 26.

As in the example of FIG. 1, medical vapor delivery system 27 of FIG. 2 may include a liquid supply 14. Liquid supply 14 may contain a liquid that is compatible with the body such as water, saline, Ringer's solution or the like. Again, the liquid may also contain other substances such as salt, iodine, chemotherapeutic agents, or the like for vaporous delivery to tissue site 20. A pump 34 may serve to circulate liquid from liquid supply 14 through liquid supply conduit 36 to surgical instrument 26.

Liquid supply 14 and liquid supply conduit 36 may or may not be insulated, as the liquid generally does not necessarily require pre-heating prior to delivery to surgical instrument 26. Accordingly, liquid supply conduit 36 may be constructed of substantially non-insulative materials such as rubber tubing, silicone tubing, and the like. In some embodiments, however, the liquid from liquid supply 14 may be pre-heated to aid in ready generation of vapor within surgical instrument 26. Like surgical instrument 18 of FIG. 1, surgical instrument 26 may take the form of any of a variety of instruments such as a catheter, forceps, hemostasis wand, and the like.

Surgical instrument 26 may be constructed in a manner to allow RF generator 28 to heat the liquid pumped from liquid supply 14. For example, leads 30, 32 may be coupled to conductors within surgical instrument 26. The conductors may be coupled to terminals that reside within a liquid reservoir, i.e., a chamber, within surgical instrument 26. The reservoir may be coupled to a distal end of conduit 36 to receive the liquid from liquid supply 14. The flow of RF current across the terminals and through the liquid causes heating and evaporation of the liquid. In some embodiments, electrical current in different frequency ranges, including dc current, may be used to heat the liquid within surgical instrument 26.

The vapor created from heating the liquid within the chamber is emitted from a distal port in surgical instrument 26 and applied to tissue site 20 in a vapor spray 22. Vapor spray 22 delivers thermal energy to tissue site 20. As described with reference to FIG. 1, the thermal energy delivered by vapor spray 22 heats the tissue site rapidly. Internal generation of vapor within surgical instrument 26 may be advantageous in terms of energy efficiency. For example, in comparison to delivery of vapor via an external vapor conduit, internally generated vapor is less susceptible to condensation and loss of energy.

When the temperature of human tissue is increased, it undergoes a series of changes. TABLE 1 below shows the effects of temperature on tissue.

TABLE 1

| TEMPERATURE ° C. | EFFECT ON TISSUE |
|---|---|
| 37-40 | No significant effect. |
| 41-44 | Reversible cell damage for exposure time less than several hours. |
| 45-49 | Cell damage becomes reversible at exceedingly short intervals. |
| 50-69 | Irreversible cell damage - ablation necrosis. |
| 70 | Threshold temperature for shrinkage of tissue; some collagen |
| | hydrogen bonds break at 60-68° C.; those with cross linkages |
| | break at 75-80° C. |
| 70-99 | Range of coagulation. Hemostasis due to shrinkage of blood |
| | vessels. |
| 100-200 | Desiccation as fluid is vaporized. Dependent on the length of |
| | time during which heat is applied, carbonization may occur, and |
| | at higher temperatures, occurs quickly. |
| >200 | Charring of tissue glucose. |

Referring to the example of FIG. 2, controller 24 can adjust the amount of current delivered to surgical instrument 18 by RF generator 28, in turn controlling the temperature and evaporation of liquid delivered from liquid supply 14. In this manner, controller 24 may further control the temperature of vapor stream 22, and the desired effects of the surgical procedure, i.e., ablation, cauterization, shrinkage, or the like.

The heat transfer process between vapor spray 22 and tissue site 20 may be approximated by applying thermodynamic theory for a simple two-phase mixing process. If steam at 100° C. and 101.35 kPa (atmospheric pressure) is injected into a known mass of tissue at 40° C., the vapor mass, steam in this case, required to reach a target temperature, 70° C. in this example, can be represented by the following mass-energy balance equation:

mₜᵢₛₛᵤₑ*ΔHₜᵢₛₛᵤₑ=mₛₜₑₐₘ*ΔHₛₜₑₐₘ (1)

where m is mass and ΔH is the change in energy or enthalpy. Equation (1) assumes that the steam quality, which is defined as the amount of saturated water vapor in the steam, is 100%, i.e. no liquid water in the steam. Equation (1) further assumes that no heat is lost. Assuming an experimentally determined heat capacity for the tissue of Cₚ₋ₜᵢₛₛᵤₑ=0.91 cal/(g.° C.), and a heat capacity for water of C_{p-water}=1.0 cal/(g.° C.), the change in enthalpy for tissue and steam is calculated in the equations below:

ΔHₜᵢₛₛᵤₑ=(T_{final}−Tᵢₙᵢₜᵢₐₗ)*Cₚ₋ₜᵢₛₛᵤₑ (2)

ΔHₜᵢₛₛᵤₑ=(70−40) (0.91) (3)

ΔHₜᵢₛₛᵤₑ=27.3 cal/g (4)

ΔHₛₜₑₐₘ=538+(T_{final}−Tᵢₙᵢₜᵢₐₗ)*Cₚ₋ₜᵢₛₛᵤₑ (5)

ΔHₛₜₑₐₘ=538+(70−40) (1.0) (6)

ΔHₛₜₑₐₘ=568 cal/g (7)

The change in enthalpy of the steam includes the energy released as the steam changes phases. Using the values from equations (2)−(7) in the energy balance equation (1) above yields

mₛₜₑₐₘ=mₜᵢₛₛᵤₑ(0.048)

Notably, the steam mass required to increase the temperature of a tissue site is extremely small in proportion to the mass of the tissue. For example, 1 gram of steam can heat nearly 20 grams of tissue to the target temperature of 70° C. in this example.

Table 2 indicates the amount of steam required to heat 1 gram of various tissues from an initial temperature of 20° C. or 40° C. to various final temperatures. The numbers in Table 2 are based in part on experimentally determined heat capacities for epidermis, dermis, fat, and muscle taken from A. R. Moritz, M.D. and F. C. Hendriques, Jr., Ph.D., Studies of thermal injury: the relative importance of time and surface temperature in the causation of cutaneous burns,” American Journal of Pathology, 1947. The values in the table 2 are also calculated using the specific heat and latent heat of vaporization of steam.

TABLE 2

| | Mass of | Mass of | Mass of | Mass of |
|---|---|---|---|---|
| | steam | steam | steam | steam |
| Temperature | required | required | required | required |
| Rise (° C.) of 1 | to heat | to heat | to heat | to heat |
| gram of tissue | epidermis (g) | dermis (g) | fat (g) | muscle (g) |
| 40 to 50 | 0.015 | 0.013 | 0.009 | 0.015 |
| 40 to 60 | 0.030 | 0.027 | 0.019 | 0.031 |
| 40 to 70 | 0.045 | 0.041 | 0.029 | 0.048 |
| 40 to 80 | 0.062 | 0.055 | 0.039 | 0.065 |
| 40 to 90 | 0.078 | 0.070 | 0.050 | 0.083 |
| 20 to 50 | 0.044 | 0.039 | 0.028 | 0.046 |
| 20 to 60 | 0.059 | 0.053 | 0.038 | 0.063 |
| 20 to 70 | 0.076 | 0.068 | 0.048 | 0.080 |
| 20 to 80 | 0.092 | 0.083 | 0.059 | 0.098 |
| 20 to 90 | 0.110 | 0.098 | 0.070 | 0.116 |

As indicated in Table 2 above, approximately 0.015 g of steam is sufficient to heat 1 g of muscle tissue from 40° C. to 50° C. As another example, to heat fat tissue from 20° C. to 90° C., approximately 0.070 g of steam is required. In general, Table 2 demonstrates that very small amounts of steam are capable of transferring very large amounts of thermal energy to a tissue site. Accordingly, steam or other vaporous media can provide a very effective tool in surgical procedures such as ablation, hemostasis, tissue shrinkage, tissue sealing, or the like. In operation, the volume and temperature of steam or other vaporous media can be controlled in view of the relationships represented in Table 2 to cause desired tissue effects in different types and volumes of tissue.

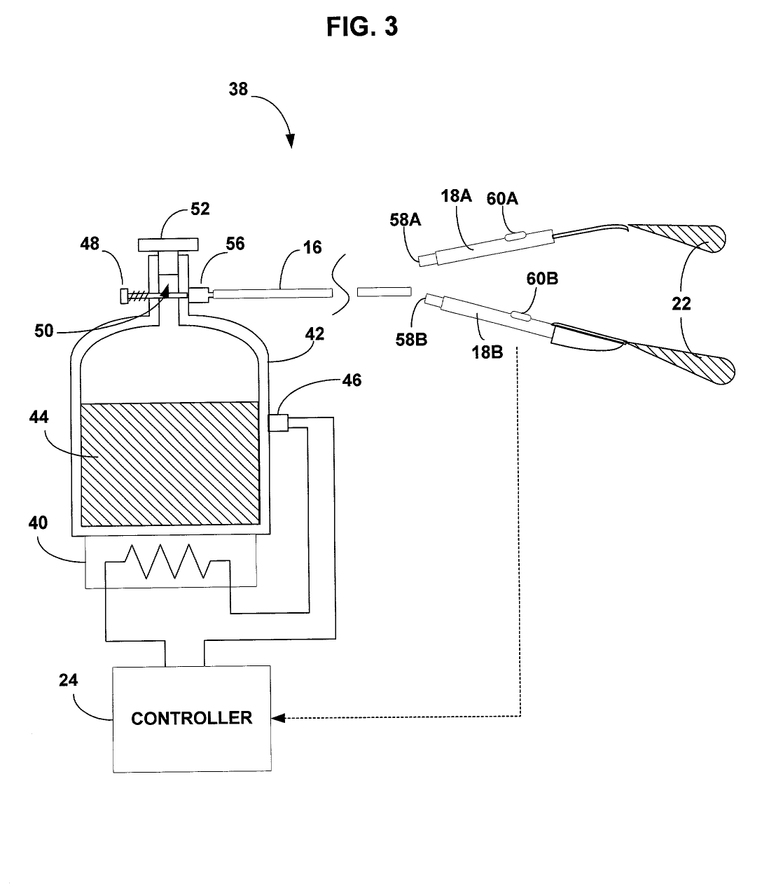

FIG. 3 is a schematic diagram illustrating an exemplary embodiment of a medical vapor delivery system 38 comprising a resistive heater 40 to generate vapor. Resistive heater 40 is located proximate a pressure vessel 42. System 38 may conform substantially to system 10 of FIG. 1. Vessel 42 contains a liquid 44. Resistive heater 40 heats vessel 42, in turn heating liquid 44. Accordingly, liquid 44 heats and begins to evaporate. As liquid 44 begins to evaporate, the vapor inside vessel 42 causes an increase in pressure inside of the vessel. The pressure inside of vessel 42 can be kept fairly constant by providing a thermal switch 46 that controls resistive heater 40. When liquid 44 reaches a target temperature of medical vapor delivery system 38, thermal switch 46 shuts off resistive heater 40. The vapor created in pressure vessel 42 may be released via a control valve 48. As the vapor exits vessel 42, vessel 42 experiences a pressure drop. The pressure drop of vessel 42 results in a reduction of temperature. The reduction of temperature is measured by thermal switch 46 and resistive heater 40 is turned back on to heat liquid 44. In one embodiment, the target temperature of vessel 42 may be set to approximately 108° C., providing a continuous supply of vapor. As the vapor is released, it undergoes a pressure drop, which reduces the temperature of the vapor to a range of approximately 90-100° C.

As liquid 44 in vessel 42 evaporates and the vapor exits vessel 42, the amount of liquid 44 slowly diminishes. Liquid 44 may be added to vessel 42 via a fill hole 50. To add liquid 44 into fill hole 50, fill plug 52 must be removed. After adding the necessary liquid 44, fill plug 52 must be placed back into fill hole 50 in order to prevent the escape of the vapor during the evaporation process.

Vapor flows from vessel 42 to a surgical instrument 18 via a vapor conduit 16. In FIG. 3, for purposes of illustration, surgical instrument 18 is shown to be either a scalpel 18B or a hemostasis wand 18A. Vapor conduit 16 couples to vessel 42 via a fluid connector 56. When control valve 48 is open, vessel 42 is in fluid communication with vapor conduit 16 via connector 56. Again, as mentioned with respect to the example of FIG. 1,vapor conduit 16 may be insulated in order to prevent an appreciable decrease in temperature of the vapor as it travels from vapor conduit 16 to surgical instrument 18. Vapor conduit 16 must be capable of handling the pressure produced inside pressure vessel 42. The pressure produced in vessel 42, using the target temperature of 108° C., may be on the order of 25 pounds per square inch (psi) (1.72 bars).

Vapor conduit 16 may be a thick walled pipe or a dual walled pipe. Vapor conduit 16 may be constructed from silicone, latex tubing, or the like, and may be flexible. Surgical instrument 18 may be a catheter, forceps, hemostasis wand, scalpel, or the like. Vapor conduit 16 couples to surgical instrument 18 via a connector interface 58. Connector interface 58 receives vapor conduit 16, placing it in fluid communication with an inner lumen of surgical instrument 18. Connector interface 58 and the distal end of vapor conduit 16 may be realized by any of a variety of conventional fluid connection arrangements, e.g., luer lock fittings, ball valve fittings, or the like. In this manner, surgical instrument 18 may be selectively connected to or removed from conduit 16, enabling a variety of different surgical instruments to be used for different procedures within system 38.

Surgical instrument 18 may further include a control switch 60, identified by reference numerals 60A, 60B of surgical instruments 18A, 18B, respectively, in FIG. 3. Control switch 60 may serve to turn vapor spray 22 on and off. For example, control switch 60 may physically open and close a valve that controls emission of vapor stream 22 from a distal region of surgical instrument 18. Switch 60 may be configured to control other attributes of the vapor such as direction, flow, pressure, volume, spray diameter, or the like. Instead of, or in addition to, physically controlling attributes of the vapor, switch 60 may electrically communicate with a controller 24. Controller 24 controls the resistive heater 40, which in turn controls attributes of the vapor, in response to actuation of switch 60 by a surgeon. In addition, controller 24 may control valves or pressure regulators associated with conduit 16 or vessel 42.

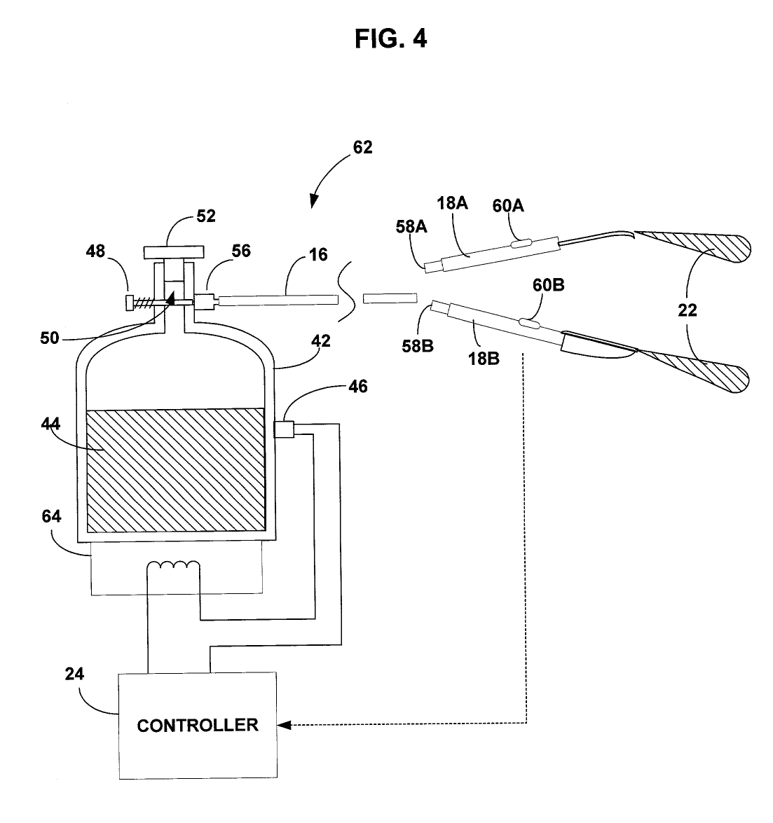

FIG. 4 is a schematic diagram illustrating another exemplary embodiment of a medical vapor delivery system 62. System 62 conforms substantially to system 38 illustrated in FIG. 3, but incorporates a radio frequency (RF) heater 64 instead of a resistive heater 40. RF heater 64 may heat liquid 44 more quickly than resistive heater 40.

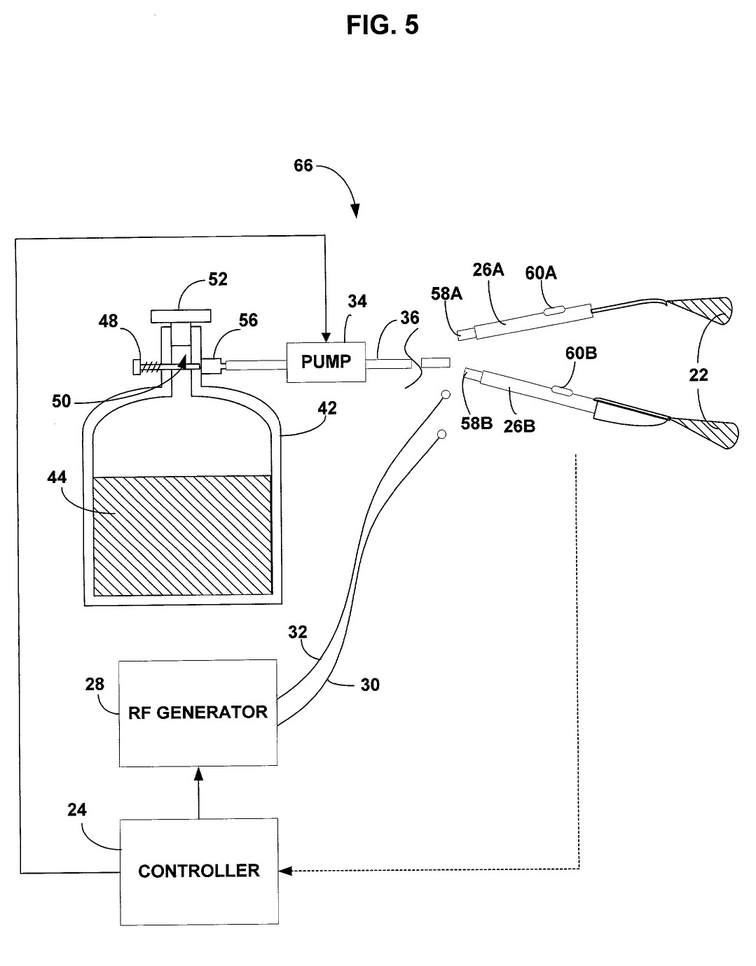

FIG. 5 is a schematic diagram illustrating an exemplary embodiment of a medical vapor delivery system 66 that generates a vaporous medium internal to surgical instrument 26. Surgical instrument 26 may be coupled to a liquid supply conduit 36 via a connector interface 58, identified by reference numerals 58A and 58B in FIG. 5. Surgical instrument 26 also may be coupled to a radio frequency generator 28 via leads 30, 32. In the example of FIG. 5, both a vapor scalpel instrument 26B and a vapor wand 26A are shown.

As in the examples of FIGS. 3 and 4, medical vapor delivery system 66 of FIG. 5 includes a vessel 42 that contains a liquid 44. System 64 further may include a pump 34, which draws liquid 44 from vessel 42 and transports liquid 44 to surgical instrument 26 via liquid supply conduit 36. Connector 56 receives liquid supply conduit 36, placing it in fluid communication with vessel 42. Surgical instrument 26 of FIG. 5 may be constructed in a manner to allow RF generator 28 to heat liquid 44 pumped from vessel 42, as described with reference to FIG. 2. For example, leads 30, 32 may be coupled to conductors within surgical instrument 26.

The conductors may be coupled to terminals that reside within a liquid reservoir, i.e., chamber, within surgical instrument 26. The reservoir may be coupled to conduit 36 to receive liquid 44 from vessel 42. The flow of RF current across the terminals and through liquid 44 causes heating and evaporation of liquid 44. The vapor created from heating liquid 44 is emitted from surgical instrument 26 and applied to a targeted tissue site in a vapor spray 22. Vapor spray 22 delivers thermal energy to the targeted tissue site. The thermal energy delivered by vapor spray 22 heats the tissue site rapidly.

Surgical instrument 26 may further include a switch 60. Switch 60 may serve to turn vapor spray 22 on and off. For example, switch 60 may physically open and close a valve that controls emission of vapor stream 22 from a distal region of surgical instrument 26. Switch 60 may be configured to control other attributes of the vapor such as direction, flow, pressure, volume, spray diameter, or the like. Instead of, or in addition to, physically controlling attributes of the vapor, switch 60 may electrically communicate with a controller 24. Controller 24 controls RF generator 28, which in turn controls attributes of the vapor, in response to actuation of switch 60 by a surgeon. For example, controller 24 may increase the potential difference, duty cycle, or frequency of the signal between leads 30, 32 extending from RF generator 28. In addition to controlling RF generator 28, controller 24 may control pump 34. For example, controller 24 may increase the flow of liquid 44 from vessel 42 in order to create a larger amount of vapor. Controller 24 may also control whether pump 34 is on or off.

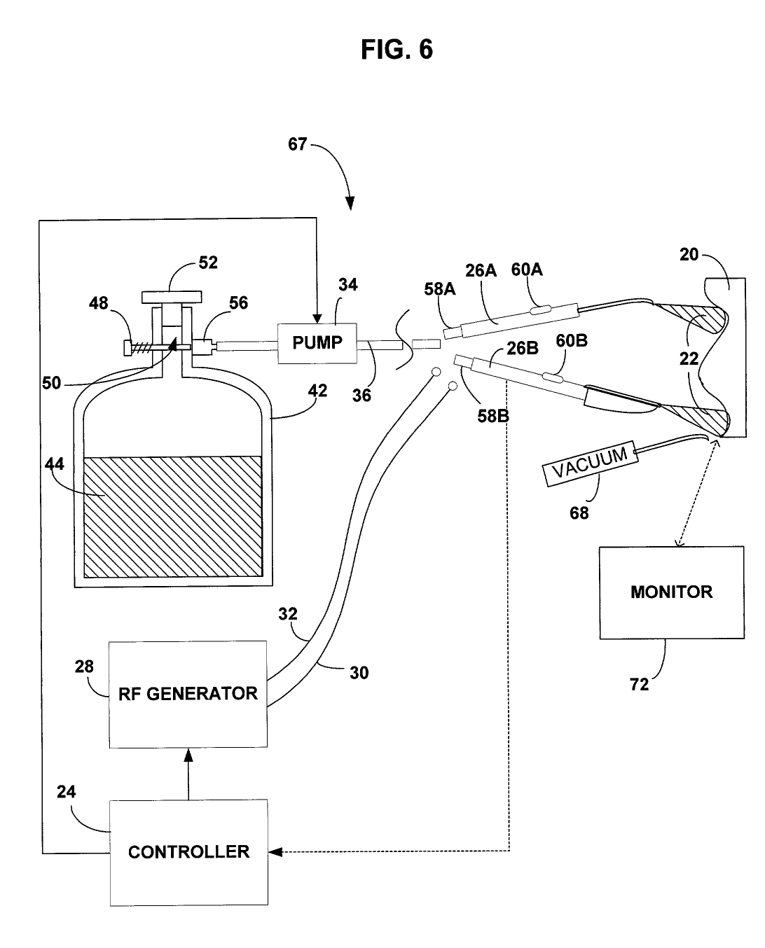

FIG. 6 is a schematic diagram illustrating another medical vapor delivering system 67 that generates a vaporous medium internal to surgical instrument 26. System 67 conforms substantially to system 66 of FIG. 5, but may further include a vacuum 68 for removal of excess fluids or tissue residue. Vacuum 68 may be external to surgical instrument 26 as shown in FIG. 6 or it may be internal to surgical instrument 26. If vacuum 68 is internal to surgical instrument 26, surgical instrument may include an internal lumen for recovery of fluids or other process residue and delivery of the residue to a waste container.

System 67 may further include a monitoring system 72. Monitor system 72 may allow the monitoring of the application of vapor spray 22 to tissue site 20. Many vapors, including steam, are hyperechoic, permitting visibility in ultrasound images. Monitor system 72 may be an ultrasound imaging device, a magnetic resonance imaging (MRI) device, a photooptic densitometer, a camera or the like. A physician may use monitor system 72, for example, to view the proper positioning of surgical instrument 26 relative to tissue site 20 and the progress of a surgical procedure. In the event a camera is used for visual imaging, monitor system 72 also may include an illumination device that illuminates tissue site 20. Ultrasound imaging may be provided by an external ultrasound transceiver, or an internally deployed probe. MRI imaging may be provided by an external MRI generator. Camera imaging may be provided by endoscopic or laparoscopic camera probes. Fluoroscopic imaging also may be useful in monitoring the progress of a procedure or the location of target tissue, particularly if surgical instrument is capable of delivering contrast agents via vapor spray 22.

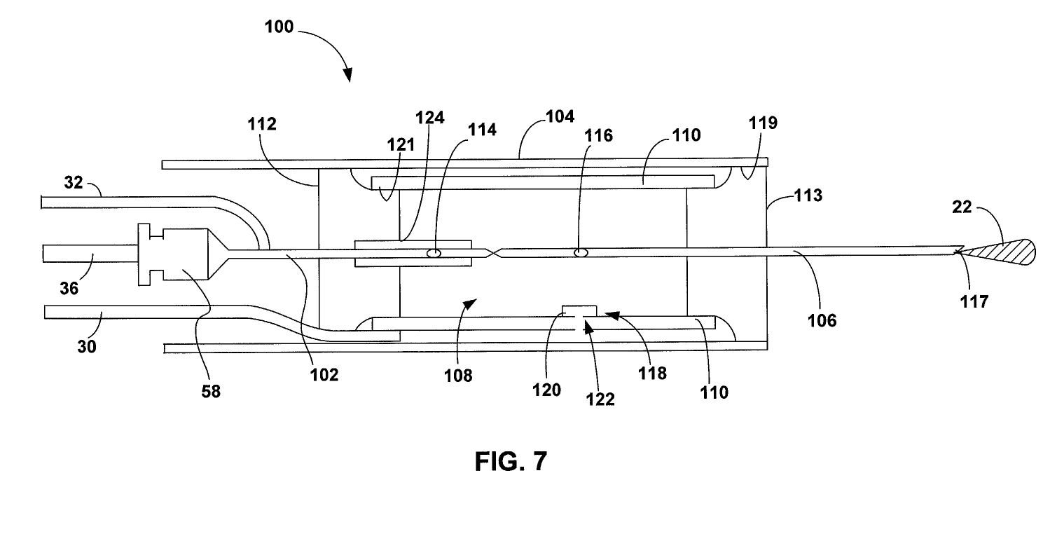

FIG. 7 is a cross-sectional side view illustrating an example of a surgical instrument 100 that generates vapor internally. Surgical tool 100 includes a housing 104, which accepts a liquid supply conduit 36 and leads 30, 32. Liquid supply conduit 36 is coupled to a connector interface 58, and receives liquid from an external liquid supply. Connector interface 58 provides fluid communication between liquid supply conduit 36and an inner liquid conduit 102.

Housing 104 may be constructed of a thermally and electrically insulated material. Liquid supply conduit 36 may be constructed of a material such as rubber, silicone, or the like. Connector interface 58 and the distal end of liquid supply conduit 36 may be realized by any of a variety of conventional fluid connection arrangements, e.g., luer lock fittings, ball valve fittings, or the like, and may be constructed from electrically insulative materials. Inner liquid conduit 102 is constructed of an electrically conductive material. The conductive material may be selected to resist rust, corrosion, or the like. As shown in FIG. 7, surgical tool 100may take the form of a needle-like ablation or cautery probe. However, other surgical tool embodiments may use internal vapor generation, such as scalpels, hemostasis wands, forceps, scalpels, or the like.

In the example of FIG. 7, housing 104 of surgical tool 100 further defines a coaxial chamber 108. A cylinder 110 and seals 112, 113, located at opposite ends of cylinder 110, define coaxial chamber 108. Compartments having non-cylindrical shapes may be substituted for cylinder 110 in some embodiments. Coaxial chamber 108 houses at least a portion of both inner liquid conduit 102 and a vapor outlet conduit 106. The portion of inner liquid conduit 102 housed in coaxial chamber 108 further includes inlet port 114. Inlet port 114 allows liquid to escape from inner liquid conduit 102 into coaxial chamber 108. Similarly, the portion of vapor outlet 106 housed in coaxial chamber 108 has an exit port 116. Exit port 116 allows the vapor to escape from coaxial chamber 108 and enter vapor outlet 106. Vapor that enters vapor outlet 106 is emitted from a distal port 117 of vapor outlet 106.

Cylinder 110 may be constructed of electrically conductive material that is resistant to rust, corrosion, and the like. Seals 112, 113 may be constructed of a material that is both electrically and thermally insulative such as rubber, silicone, or the like. Seals 112, 113 may form a compression fit with inner wall 119 of housing 104 and an inner wall 121 of cylinder 110 to seal chamber 108 against leakage of liquid.

In operation, liquid enters surgical instrument 100 via inner liquid conduit 102. The liquid travels through inner liquid conduit 102 and enters coaxial chamber 108 via inlet port 114. Lead 30 is electrically coupled to cylinder 110. Lead 32 is electrically coupled to inner liquid conduit 102. The liquid that fills coaxial chamber 108 via inner liquid conduit 102 completes an electrical circuit between cylinder 110 and inner liquid conduit 102. Upon application of RF current across electrodes 30, 32, chamber 108 functions as a radio frequency generator that heats its contents.

Surgical instrument 100 may further include an electrically insulating sheath 124 that fits loosely over inner liquid conduit 102. Electrically insulating sheath 124 can be provided to increase the electrical path length through the liquid in order to better match the impedance of an RF generator coupled to leads 30, 32. Other techniques for providing an acceptable impedance match may involve use of fluids with different conductivity characteristics, and incorporation of dielectric materials such as porous or pathway lengthening materials within cylinder 110.

RF energy is converted into thermal energy via “ohmic” impedance. Accordingly, it is necessary to have resistance to produce thermal energy to convert a liquid to a vapor. RF generators have inherent capabilities of outputting RF power. If the impedance is too low, most of the RF energy is returned to the generator. Most conventional RF generators perform best between 50 ohms and 1000 ohms of resistance. Therefore, it is important to provide a sufficient amount of RF electrical impedance is provided between the leads 30, 32. The geometric dimensions of the RF electrodes 30, 32, the materials used to make the electrodes, and the fluid and materials between the leads affect the impedance to RF flow.

Fluids with different impedances can be used to optimize the combination of RF generator, the electrodes, and the fluids or other materials in order to optimize the energy conversion. Solutions with greater ionizing potential and higher concentrations generally will exhibit a greater electrical conductivity. If greater distances are used between RF leads 30, 32, it may be desirable to increase the conductivity of the solution. Conversely, if the RF vapor generating cylinder 110 has a small diameter, it may be necessary to decrease the electrical conductivity (increase the impedance).

The altering of impedance between the RF leads 30, 32 can also be accomplished by adding physical materials such as plastic or ceramic beads or a sintered material between the electrodes. These materials can increase the path length for the fluid to travel between the electrodes and increase the electrical impedance in the process. This increase in path length can also provide for greater dispersion of RF current to increase the effectiveness and efficiency of producing vapor.

The radio frequency generator formed within chamber 108 produces heat in the area of the highest current density, which generally occurs at the longitudinal axis of coaxial chamber 108. The heat produced by the RF energy evaporates the liquid within chamber 108, and increases the internal pressure within the chamber. The pressure within coaxial chamber 108 causes the vapor to exit coaxial chamber 108 via exit port 116. The vapor enters vapor outlet 106 via exit port 116 and is applied to a tissue site via distal outlet 117 as vapor spray 22. In general, the flow of the vapor produced within chamber 108 is a function of the flow rate of liquid into surgical instrument 100 via inlet port 114, given a fixed amount of RF energy.

In some embodiments, inlet port 114 may be coupled to a check valve that prevents backflow of vapor from chamber 108 into inner liquid conduit 102. Flow rate can be controlled with a pump to meter the amount of vapor produced by surgical instrument 10. Also, the RF energy applied within chamber 108 may be controlled to adjust the amount of vapor produced by surgical instrument 10. The coaxial design of coaxial chamber 108 allows the electrode contact surface between the liquid and the inner fluid conduit 102 to increase as the liquid pump flow rate increases. An RF generator 28 coupled to leads 30, 32 can be controlled to adapt to the level of power needed as the impedance of coaxial chamber 108 drops.

Surgical instrument 100 also may incorporate a one-way valve 118 located on cylinder 110. One-way valve 118 prevents the backward flow of liquid, such as blood, into vapor outlet 106 and coaxial chamber 108. When surgical instrument 100 is shut off, a pressure drop occurs due to the cooling of the vapor inside chamber 108. Valve 118 can be configured to open when the pressure inside coaxial chamber 108 goes below atmospheric pressure. The pressure inside coaxial chamber 108 is thereby equalized, preventing the backward flow of blood or the like. Valve 118 may be constructed of a flexible ring 120 mounted about a hole 122 formed in cylinder 110. Flexible ring 120 may be constructed of a flexible material such as silicone rubber or the like. To avoid contamination, a small particle filter may be disposed over or within valve 118. Alternatively, valve 118 may be coupled to a sterile gas source, e.g., a CO2 reservoir. In this manner, introduction of unfiltered air into cylinder 110 can be avoided.

In some embodiments, vapor outlet 106 may have a concentric, double lumen construction in which an inner lumen has an outlet orifice that delivers vapor to the tissue site, and an outer lumen allows vapor to flow counter-current in the outer lumen. This countercurrent exchange mechanism may be effective in reducing thermal loss in vapor outlet 106 and can reduce condensation, thereby maintaining vapor quality. Countercurrent vapor flow could be controlled by either a valve at the inner lumen opening or at the outer lumen outlet, providing a dual purpose of controlling pressure and improving vapor quality. A pressure sensor such as a piezoelectric sensor could also be installed in the vapor circuit to measure and control outlet pressure.

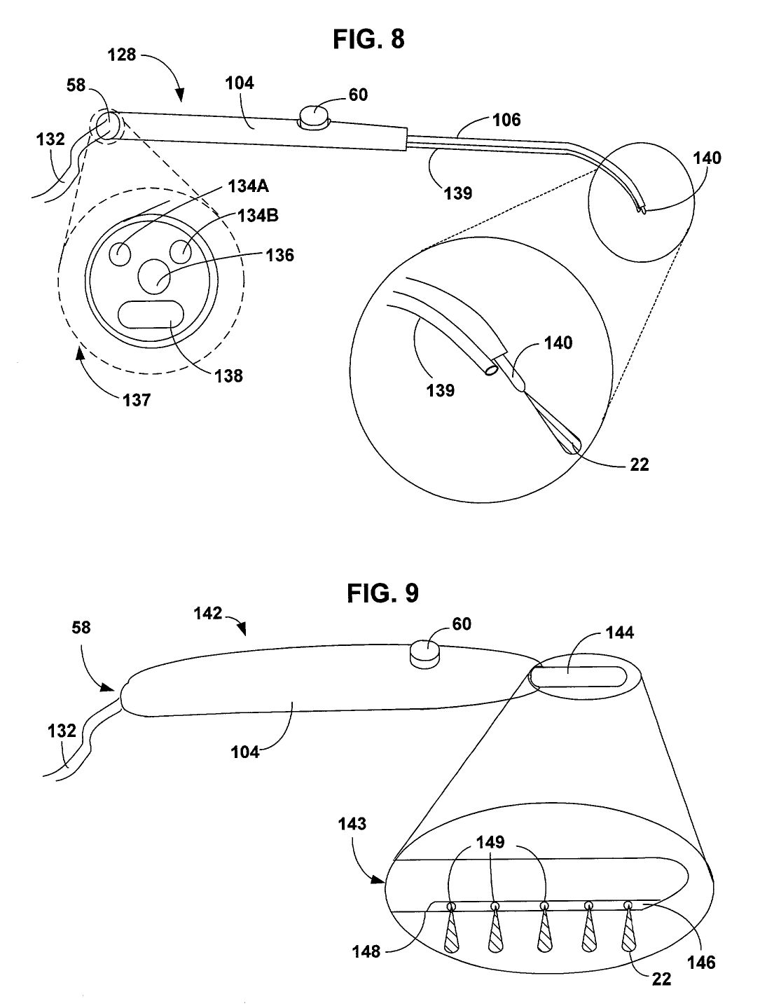

FIGS. 8-17 are schematic diagrams illustrating various surgical instruments that may be used to deliver a vaporous medium to a tissue site, in accordance with the invention. FIG. 8 is a schematic diagram illustrating a vapor hemostasis wand 128. Hemostasis wand includes a housing 104 that may house an internal vapor generator as described in FIG. 7. Housing 104 includes a connector interface 58 that accepts a conduit 132. Conduit 132 may be a vapor delivery conduit supplying vapor to hemostasis wand 128 from an external vapor generator. Alternatively, conduit 132 may include a liquid delivery conduit and RF generator leads for internal vapor generation within wand 128 in a manner substantially as described above with reference to FIG. 7. Conduit 132 also may include a vacuum conduit in order to remove tissue residue from a tissue site. Enlarged portion 137 of FIG. 8 shows front view of connector interface 58. Connector interface 58 of hemostasis wand 128 has a pair of lead receptacles 134A and 134B to a pair of RF leads, a liquid receptacle 136 for fluid communication with a liquid conduit, and a vacuum receptacle 138 for communication with a vacuum pump to remove fluidized tissue residue from the tissue site.

Hemostasis wand 128 may also include a control switch 60. Control switch 60 may serve to turn a vapor spray 22 on and off. For example, switch 60 may physically open and close a valve that controls emission of vapor stream 22 from a distal region of surgical instrument 54. Control switch 60 may be configured to control other attributes of the vapor such as direction, flow, pressure, volume, spray diameter, or the like. Instead of, or in addition to, physically controlling attributes of the vapor, control switch 60 may electrically communicate with a controller 24 (not shown in FIG. 8). Controller 24 may control a heater, which in turn controls attributes of the vapor, in response to actuation of control switch 60 by a surgeon. Controller 24 may also control a pump, which pumps liquid into hemostasis wand 128. Furthermore, the controller 24 may control operation of an RF generator if vapor is generated internally to instrument 128.

An enlargement of a tip 140 of hemostasis wand 128 shows a vapor spray 22 emitted from tip 140. Tip 140 extends from the distal end of a vapor outlet conduit. Tip 140 may be thermally insulated to prevent burning of tissue upon accidental contact of tip 140 and the tissue site. A vacuum line 139 also may be provided adjacent tip 140. Vacuum line 139 may be coupled for fluid communication with vacuum receptacle 138.

FIG. 9 is a schematic diagram illustrating a vapor cutting hemostat 142. Cutting hemostat 142 includes a housing 104 that may house an internal vapor generator as described in FIG. 7. Housing 104 accepts a conduit 132, e.g., carrying liquid and RF leads, via connector interface 58. Cutting hemostat 142 may also include a control switch 60. Cutting hemostat 142 further includes a cautery blade 144. Thus, cutting hemostat 142 may be constructed to function as both a hemostat and a scalpel. Enlarged view 143 illustrates cautery blade 144 in greater detail. As shown, cautery blade 144 may include a vapor orifice plane 146. Vapor orifice plane 146 emits a vapor spray 22 from a number of small orifices 149 distributed along the length of orifice plane 146 to a tissue site to cauterize the tissue site as a cutting edge 148 cuts the tissue site. Cutting edge 148 may cut the tissue like a conventional scalpel or apply RF current between electrodes formed on the cutting edge. Advantageously, in the event RF is used, the vapor may be generally non-conductive so as not to interfere with the RF cutting process. In other embodiments, the fluid that is vaporized may be loaded with a conductive material, e.g., sodium, to enhance energy transfer and promote deeper tissue heating for more effective hemostasis.

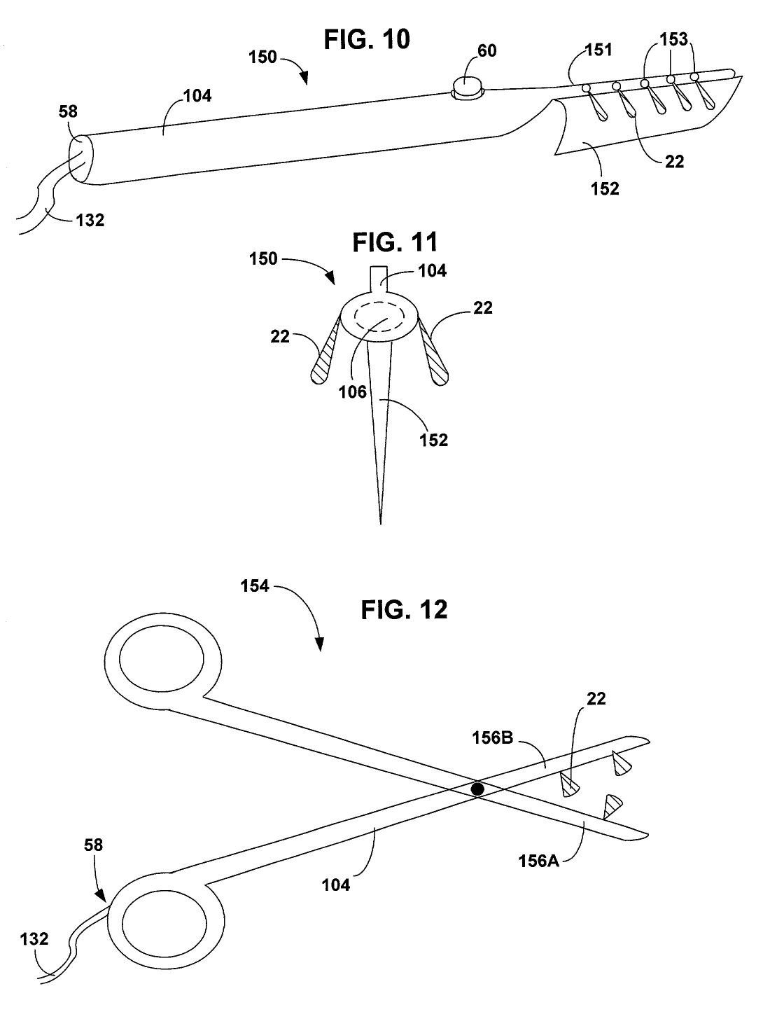

FIG. 10 is a schematic diagram illustrating a vapor scalpel 150 that may be configured for use in accordance with the invention. Vapor scalpel 150 includes a housing 104 that may house an internal vapor generator as described with respect to the example of FIG. 7. Housing 104 accepts a conduit 132 via connector interface 58. Vapor scalpel 150 may also include a control switch 60. Vapor scalpel 150 further includes a cutting blade 152, which is used to cut a tissue site. Housing 104 may be shaped to extend into a narrow distal tip portion 151 that holds a cutting blade 152. Distal tip portion 151 of housing 104 may have several orifices 153 that emit a vapor spray 22. Vapor spray 22 can be delivered along the cutting path of blade 152 to coagulate blood and cauterize tissue on both sides of the cut, stopping bleeding during the cutting of tissue and blood vessels.

FIG. 11 is a schematic diagram illustrating a front profile view of vapor scalpel 150 of FIG. 10. The view of vapor scalpel 150 shows a vapor outlet 106 that travels down the distal tip portion 151 of housing 104 of vapor scalpel 150. Vapor scalpel 150 emits vapor sprays from small orifices 153 in vapor outlet 106 and housing 104. As shown in FIG. 11, orifices 153 expel vapor spray 22 out both sides of cutting blade 152. As an alternative to discrete orifices 153, vapor scalpel 150 may include a porous material that exudes vapor uniformly in the region of cutting blade 152. For example, a sintered material may be used for the base of cutting blade 152 with a length of solid blade material attached. The sintered material may provide for a uniform spray of hot vapor to increase effective hemostasis.

FIG. 12 is a schematic diagram illustrating a vapor coagulating scissors 154. Vapor coagulating scissors 154 includes a housing 104 that may house an internal vapor generator as described in FIG. 7. Housing 104 may be shaped in a manner to function as a handle of a scissors. Housing 104 accepts a conduit 132 near the base of one of the scissor handles via a connector interface 58. Coagulating scissors 154 may also include a control switch 60 (not shown in FIG. 12). Coagulating scissors 154 further includes a pair of cutting blades 156A, 156B. A set of vapor sprays 22 is emitted near cutting blades 156. Vapor sprays 22 coagulate the tissue as cutting blades 156 cut the tissue.

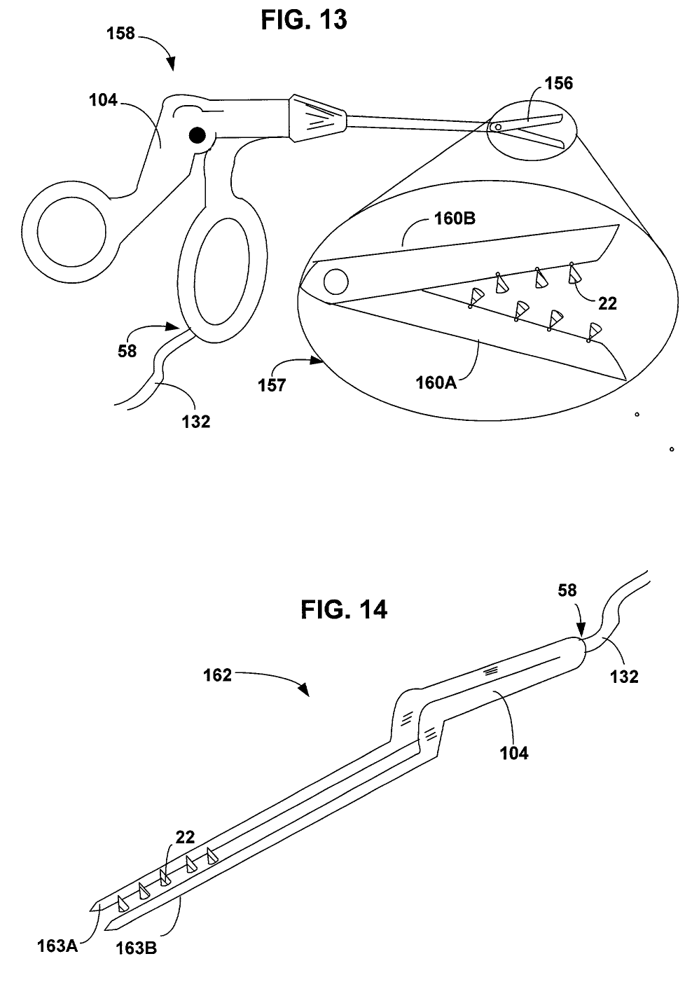

FIG. 13 is a schematic diagram illustrating a laparoscopic vapor coagulating scissors 158. Laparoscopic vapor coagulating scissors 158 conforms substantially to the vapor coagulating scissors 154 described above with reference to FIG. 12. Laparoscopic vapor coagulating scissors 158 has a housing 104, however, that is shaped slightly differently from the housing of scissors 154 in order to allow for the deployment and operation of cutting blades 156 within a constricted area inside a patient. An enlargement of cutting blades 156 shows a set of vapor sprays 22 emitted near cutting blades 156. Again, the vapor sprays 22 coagulate the tissue site as the cutting blades 156 cut the tissue site.

FIGS. 14-17 are schematic diagrams illustrating different types of forceps that may be configured to emit vapor for transfer of thermal energy in accordance with the invention. FIG. 14 is a schematic diagram illustrating an exemplary embodiment of a vapor forceps 162. Forceps 162 comprises a housing 104 that may contain an internal vapor generator as described in FIG. 7. Housing 104 accepts a conduit 132 near the base of the scissor handle via connector interface 58. Forceps 158 includes a pair of jaws 163A, 163B. Each jaw 163 may include a series of orifices that emit a vapor spray 22 toward the other jaw. Vapor spray 22 may heat the tissue seized between jaws 163A, 163B, and can be effective in sealing the tissue together. For example, the tissue sealing may be driven by hemostasis, aerostasis, or both.

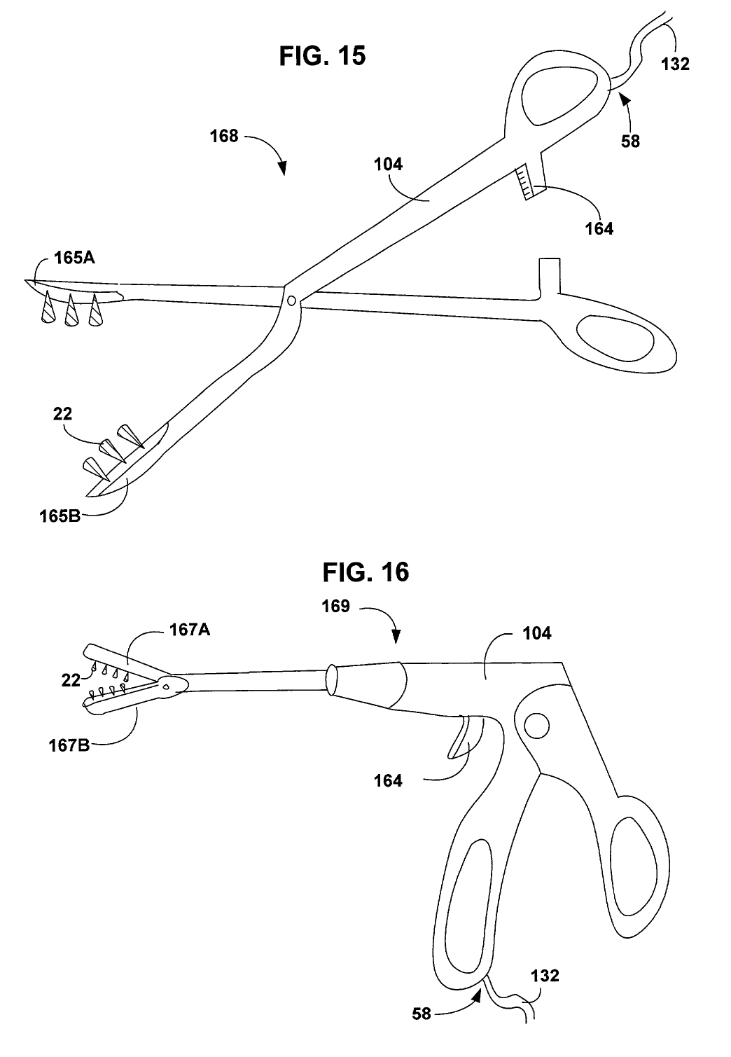

FIG. 15 is a schematic diagram of a vapor scissor forceps 168 that conforms substantially to forceps 162 described in FIG. 14. A housing 104 of the vapor forceps 168 is shaped much like a scissors, with forceps jaws 165A, 165B instead of blades. Vapor scissor forceps 168 further may include a locking mechanism 164, which allows scissor forceps 168 to be locked into place without the user continually applying pressure. Scissor forceps 168 may function as forceps 162 in FIG. 14 does, i.e. by emission of vapor spray 22 from one jaw 165A toward the other jaw 165B for tissue sealing.

FIG. 16 is a schematic diagram of a laparoscopic vapor scissor forceps 169, which conforms substantially to scissor forceps 168 described in FIG. 15. Laparoscopic scissor forceps 169 has a housing 104 that is shaped slightly different from scissor forceps 168, however, to allow for the operation of jaws 167A, 167B in a smaller area inside a patient.

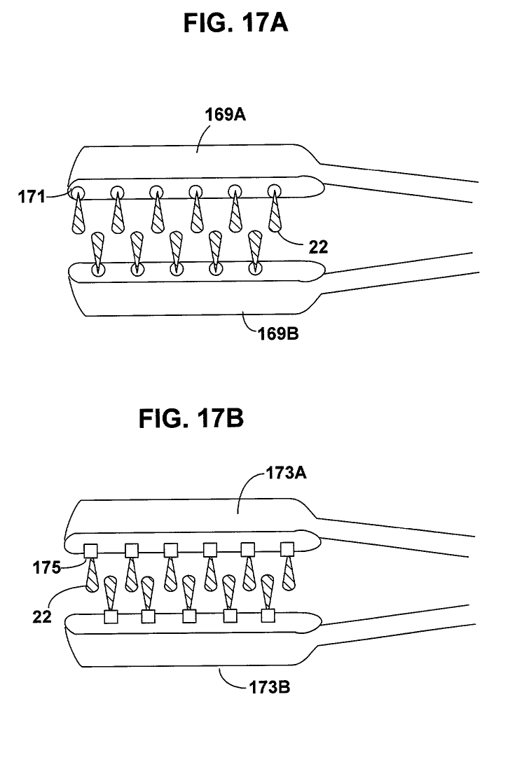

FIG. 17 is an enlarged schematic diagram illustrating jaws 169A, 169B suitable for use with the vapor forceps described in FIGS. 14-16. Jaws 169A, 169B may be generally flat and contain multiple orifices 171 for emitting a vapor spray 22 as shown in FIG. 17(A). FIG. 17(B) shows a pair of jaws 173A, 173B, which has a set of multiple protrusions 170. Each protrusion 170 has an orifice that emits a vapor spray 22. Protrusions 175 may be designed to penetrate the tissue, in turn increasing the heat transfer of the vapor.

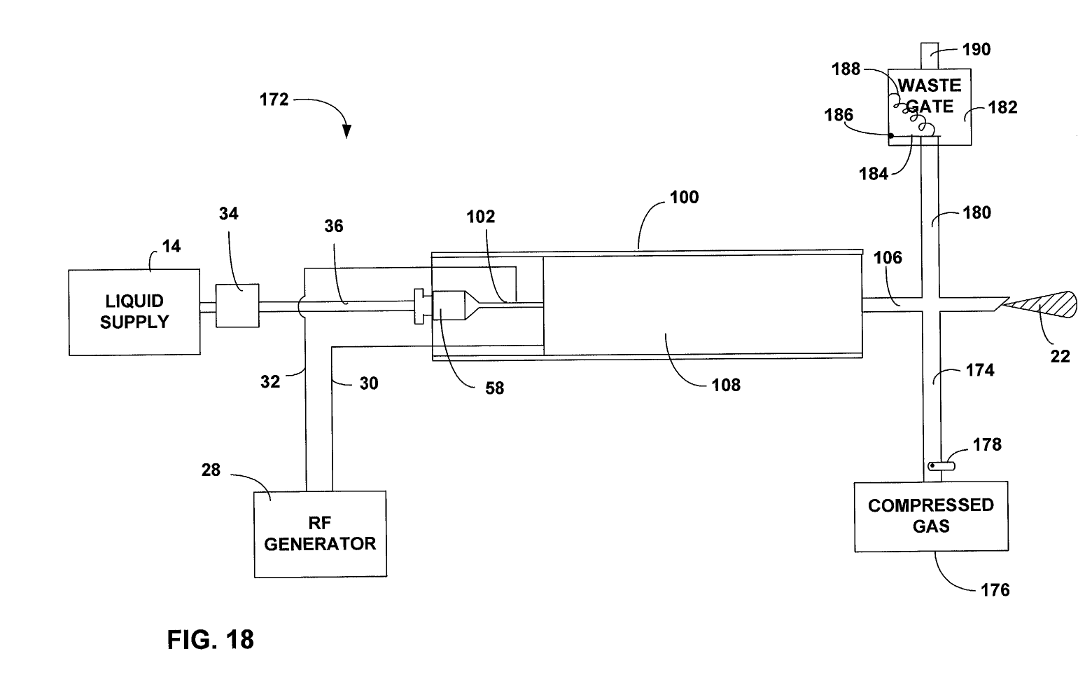

FIG. 18 is a schematic conceptual diagram of a medical vapor delivery system 172 configured to control the temperature and pressure of a vapor spray 22. Vapor medical system 172 includes a liquid supply 14 with a pump 34. Pump 34 supplies liquid from liquid supply 34 to a surgical instrument 100 via a liquid supply conduit 36. Liquid supply conduit 36 couples surgical instrument 100 via a connecting interface 58. Pump 34 may be an adjustable flow pump such as the adjustable flow peristaltic pump commonly found in surgery rooms. Liquid supply conduit 36 may be a tube-like structure made of a material such as rubber, silicone, plastic, or the like. Connecting interface 58 and the distal end of liquid supply conduit 36 may be realized by any of a variety of conventional fluid connection arrangements.

System 172 may also includes a radio frequency (RF) generator 28. RF generator 28 extends a pair of leads 30, 32 to surgical instrument 100. Lead 30 is electrically coupled to a cylinder (not shown in FIG. 18)that defines an internal chamber as described above with reference to FIG. 7. Lead 32 is electrically coupled to inner liquid conduit 102. As described in FIG. 7, the liquid in coaxial chamber 108 completes the circuit between leads 30, 32 108. As further shown in FIG. 18, a vapor outlet 106 may be coupled to a gas conduit 174. Gas conduit 174 is coupled to a pressurized gas supply 176 via a gas valve 178. Gas valve 178 may be actuated to supply pressurized gas from pressurized gas supply 176 to the interior of vapor outlet 106. The pressurized gas may be used to control the thermal energy content and temperature of vapor spray 22. Instead of, or in addition to, controlling the temperature of vapor spray 22, the pressurized gas may controllably increase the pressure of vapor spray 22.

A waste gate conduit 180 also may be coupled between vapor outlet 106 and a waste gate 182. Waste gate 182 includes a flap valve 184 coupled to a hinge 186 and a spring 188. Waste gate 182 may be used to control maximum pressure of vapor spray 22. Flap valve 184 of waste gate 182 may remain closed to allow pressure to build. If the pressure becomes too high, spring 188 will compress, causing flap valve 184 to open, relieving pressure via an escape port 190. Spring 188 may be an adjustable tension spring. The adjustability of spring 188 determines the amount of pressure needed to open flap valve 184. In some embodiments, valve 184 may be actuated by an electrical solenoid or other electromechanical actuator in response to a control signal emitted based on pressure measurements by a pressure sensor.

If the surgeon wants the pressure inside of the valve outlet 106 to be higher, adjustable spring 188 can be adjusted to increase the amount of force needed to compress spring 188. Controlling the pressure and temperature of vapor spray 22 controls the amount of thermal energy transported by the vapor to the tissue site and, in turn, controls the type of surgical procedure vapor spray 22 performs. For example, more pressure may produce a greater ablation depth. For example, increased pressure may serve to force vapor further into the interstitial spaces between tissue cells. Higher vapor pressures may be achieved by altering the diameter of the outlet orifice, e.g., to promote tissue removal versus tissue killing or alteration without removal. Also, a decrease in temperature from 80° C. to 70° C. may allow a surgeon to shrink tissue at a tissue site instead of coagulating or ablating the tissue. Precise control of temperature may permit a controllable degree of tissue shrinkage. In particular, higher temperatures cause more collagen disulfide and hydrogen bonds in the tissue to be broken, allowing increased tissue shrinkage.

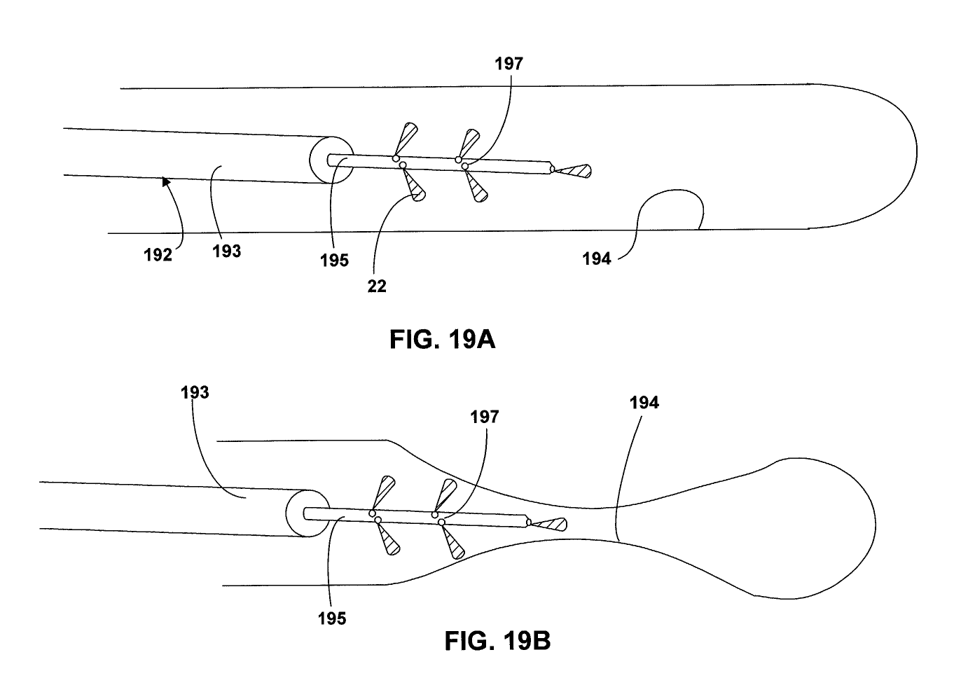

FIG. 19 is a schematic diagram illustrating a vapor catheter 192 useful for intraluminal procedures such as intraluminal shrinking. Catheter 192 emits a vapor spray 22 or several vapor sprays 22 toward a lumen wall 194. As shown in FIG. 19, catheter 192 includes a catheter body 193 and a vapor probe 195 that extends from a distal end of the catheter body. Vapor probe 195 defines orifices 197 that emit vapor spray 22 toward lumen wall 194. Lumen wall 194 may be associated with a blood vessel or other body lumen. Vapor spray 22 heats the lumen wall 194.

The temperature of lumen wall 194 begins to rise and both circumferential and longitudinal connective tissues found in the wall begin to depolymerize and shrink. Lumen wall 194 begins to collapse inwardly in a radial direction and shorten in a longitudinal direction, as shown in FIG. 19B. In this manner, lumen walls 194 may be shrunk as desired. Lumen walls 194 may even be shrunk to the point of being occluded. If a large amount of steam is used, a return line for pressure may be required. Increasing vapor pressure and volume can increase the distance of vapor travel and tissue shrinkage through the lumens. The extent of vapor travel could be estimated by monitoring temperature with a thermocouple that extends from the distal tip of endoluminal catheter 192. In general, increased pressure and increased plume size results in a greater distance of travel of the vaporous medium within a lumen, and increased shrinkage.

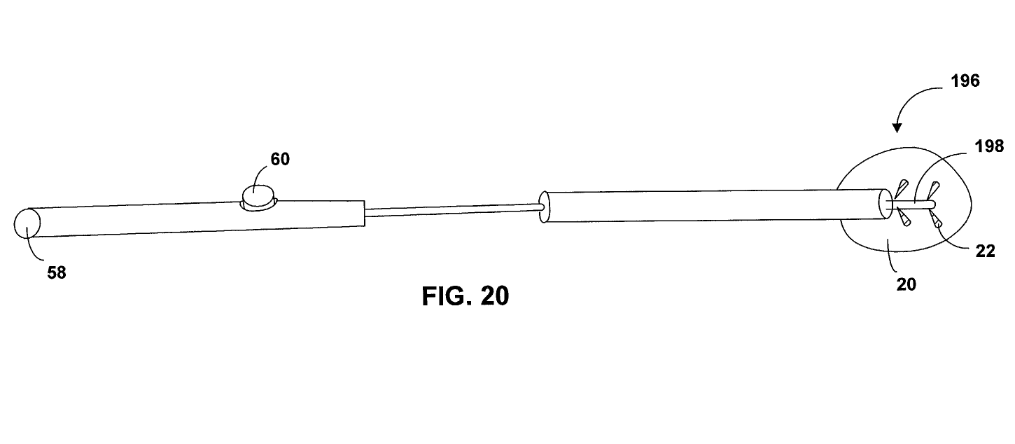

FIG. 20 is a schematic diagram illustrating an insulated vapor needle 196 being used for tissue ablation. Needle 196 may have a closed sharp tip 198 along with numerous orifices at the distal end to allow for a vapor spray 22 to be emitted. Needle 196 emits pressurized vapor sprays 22 from the distal end of needle 196 to a tissue site 20. The vapor heats the tissue at tissue site 20. Tissue site 20 is heated to a temperature that causes ablation. Similar to catheter 192 in FIG. 19, needle 196 may have a return line for pressure if a large quantity of steam is used.

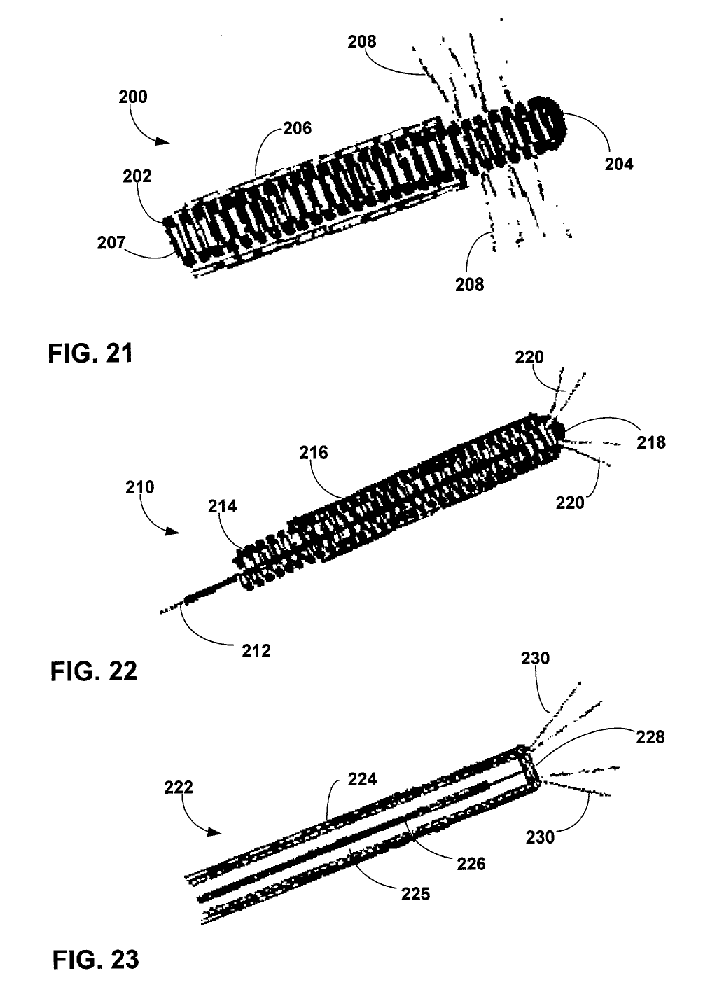

FIGS. 21-23 are schematic diagrams of catheters for delivery of a vaporous medium. The catheters of FIGS. 21-23 may be sized for a variety of endoluminal applications within a patient's body, including use within larger blood vessels in the body and smaller blood vessels in the brain. As shown in FIG. 21, a catheter 200 includes an elongated coil 202 with a distal tip 204. A flexible sheath 206 covers coil 202 and exposes distal tip 204.

A central lumen 207 transports vapor along the length of catheter 200 toward distal tip 204. In some embodiments, a miniaturized RF chamber, e.g., similar to that shown in FIG. 7, may be provided near distal tip 204 of catheter 200. In this manner, fluid can be transported along central lumen 207 and heated near distal tip 204.

In the exposed area of coil 202, the vapor 208 escapes from catheter 200 for use in shrinking, ablating, cauterizing adjacent tissue or performing other tissue procedures. The direction and volume of vapor emitted by catheter 200 may be controlled in part by selection of the shape, length and size of sheath 206. To emit vapor from only one side of catheter 200, for example, sheath 206 may be shaped to cover an opposite side of coil 202.

FIG. 22 illustrates another catheter 210 that conforms substantially to catheter 200 of FIG. 21. Catheter 210 includes a guidewire 212, a coil 214 that extends over the guidewire, and a sheath 216 that extends over the coil. In the example of FIG. 22, vapor 220 such as steam is emitted from distal tip 218 of catheter 210. As shown, sheath 216 covers much of coil 214 near distal tip 218, causing the vapor to be projected somewhat longitudinally, rather than laterally, from distal tip 218.

Catheter 222 of FIG. 23 is similar to catheters 200, 210. Catheter 22 includes a sheath 224 that covers a guidewire 226. Fluid or vapor is transported along an annular lumen 225 within catheter 222. Vapor 230 is emitted from distal tip 228. Again, the vapor may be transported along the entire length of catheter 222 or generated within an RF chamber in catheter 222.

A medical vapor delivery system, as described above in numerous embodiments herein, may be used for many different surgical applications. A vapor medical system may be used, for example, to ablate tissue, cauterize tissue, seal tissue, shrink tissue, coagulate tissue, or the like. A medical vapor delivery system can be used for applications such as, but not limited to, treatment of varicose veins, thermotherapy of benign prostatic hyperplasia (BPH), destruction of in situ prostatic cancer, urinary incontinence, treatment of uterine fibroids, cosmetic surgery, female sterilization, applications in interventional neurology and orthopedics, percutaneous ablation of tumors in the liver or kidney, and epicardial ablation to treat atrial fibrillation.

Varicose veins occur when veins become enlarged with pools of blood due to the failure to circulate blood properly. These visible and bulging veins are often associated with symptoms such as tired, heavy, or aching limbs. A vapor delivery system may be used to treat varicose veins. The vapor delivery system may use a catheter to apply a vapor spray to the lumen walls of the vein. The vapor spray transfers heat to the lumen walls of the vein causing the vein to shrink, as shown in FIG. 19.

Benign prostatic hyperplasia also may be treated using a vapor delivery system. The vapor delivery system can completely eliminate prostate tissue without severe associated morbidity. The vapor delivery system can be used with a direct endoscopic approach to perform total ablation of the prostate gland. The vapor spray from an endoscopic instrument is emitted inside of a fibrous capsule surrounding the prostate. The fibrous capsule is able to retain the high temperature vapor from the vapor delivery system. The high temperature vapor ablates the entire interior of the prostate gland while leaving the surrounding tissue undamaged. No further chemotherapy or radiation treatment may be necessary for the patient.

Urinary incontinence is the unintentional loss of urine. The vapor delivery system may treat urinary incontinence in much the same way as varicose veins. A surgical instrument such as a catheter is placed inside of the urethral and periurethral tissue sites. Vapor spray is emitted within these sites and tissue shrinkage occurs, as described in FIG. 19. The technique is minimally invasive and provides significant permanent relief.

Fibroids are benign tumors that develop in the wall of the uterus. The vapor delivery system may be used with hysteroscope access to quickly kill fibroid tissue. A vapor spray from the vapor delivery system heats the tissue site to a temperature high enough to cause ablation, as described in FIG. 20. The process may be monitored by ultrasound to ensure efficacy and safety. The procedure could be used on an outpatient or office basis and should present minimal post-operative pain.

As a further application, the vapor delivery system may be used to shrink the fallopian tube to provide female sterilization. A vapor spray may be emitted inside the fallopian tube causing it to shrink, as described in FIG. 19. The procedure for shrinking the fallopian tube may be performed as a transcervical procedure via hysteroscopy. The length of time for total occlusion of the fallopian tube would be far less than the length of time required by other techniques.

Percutaneous ablation of tumors in liver and kidney also may be accomplished via the vapor delivery system. A vapor spray may rapidly deliver a uniform killing temperature to a mass of tissue, as described in FIG. 19. The killing efficacy may be monitored by ultrasound, ensuring the ablative lesion encompasses the entire tumor without causing damage to adjacent tissue.

Atrial fibrillation is caused by aberrant pathways of electrical conduction that trigger uncoordinated atrial contractions, resulting in impaired hemodynamics. The vapor delivery system may create transmural lesions from the epicardial surface, which will effectively terminate atrial fibrillation. The procedure may be developed for open heart procedures and ultimately be adapted to minimally invasive port access procedures.

A number of embodiments of the present invention have been described. Nevertheless, it will be understood that various modifications may be made without departing from the spirit and scope of the invention. Accordingly, other embodiments are within the scope of the following claims.

1. A method comprising
generating a vaporous medium; and
delivering the vaporous medium to a tissue site of a patient to transfer thermal energy to the tissue site.

2. The method of claim 1, wherein delivering the vaporous medium includes delivering the vaporous medium via a surgical instrument.

3. The method of claim 2, further comprising generating the vaporous medium external to the surgical instrument.

4. The method of claim 2, further comprising generating the vaporous medium internal to the surgical instrument.

5. The method of claim 1, further comprising generating the vaporous medium via one of a radio frequency vapor generator and a resistive vapor heater.

6. The method of claim 1, wherein the vaporous medium includes steam.

7. The method of claim 1, further comprising controlling the vaporous medium to deliver an amount of the thermal energy sufficient to ablate tissue at the tissue site.

8. The method of claim 1, further comprising controlling the vaporous medium to deliver an amount of the thermal energy sufficient to provide hemostasis at the tissue site.

9. The method of claim 1, further comprising controlling the vaporous medium to deliver an amount of the thermal energy sufficient to cauterize tissue at the tissue site.

10. The method of claim 1, further comprising controlling the vaporous medium to deliver an amount of the thermal energy sufficient to coagulate tissue at the tissue site.

11. The method of claim 1, further comprising controlling the vaporous medium to deliver an amount of the thermal energy sufficient to shrink tissue at the tissue site.

12. The method of claim 1, further comprising controlling the vaporous medium to deliver an amount of the thermal energy sufficient to seal tissue at the tissue site.

13. The method of claim 1, further comprising controlling at least one of direction, duration, pressure, temperature, and volume of the vaporous medium.

14. The method of claim 1, further comprising monitoring the delivery of the vaporous medium.

15. The method of claim 13, further comprising adding a substance to the vaporous medium such that the substance is carried to the tissue site.

16. The method of claim 1, wherein generating the vaporous medium includes evaporating a liquid.

17. The method of claim 16, wherein the liquid includes one of water and saline.

18. The method of claim 17, wherein the liquid includes salt, iodine, lidocaine, and chemotherapeutic materials.

19. An apparatus comprising:
a supply of liquid;
a source of thermal energy to evaporate the liquid to generate a vaporous medium; and
an instrument with a vapor outlet to deliver the vaporous medium to a tissue site of a patient.

20. The apparatus of claim 19, wherein the liquid is one of water and saline.

21. The apparatus of claim 19, wherein the liquid includes salt, iodine, lidocaine, and chemotherapeutic materials.

22. The apparatus of claim 19, wherein the source of energy is one of a radio frequency heater, a resistive heater, a microwave heater, a laser heater, or a high intensity focused ultrasound heater.

23. The apparatus of claim 19, wherein the vaporous medium transfers an amount of thermal energy to the tissue site, the apparatus further comprising a controller to control the amount of thermal energy transferred by the vaporous medium.

24. The apparatus of claim 23, wherein the amount of thermal energy transferred by the vaporous medium to the tissue site is sufficient to ablate tissue at the tissue site.

25. The apparatus of claim 23, wherein the amount of thermal energy transferred by the vaporous medium to the tissue site is sufficient to provide hemostasis at the tissue site.

26. The apparatus of claim 23, wherein the amount of thermal energy transferred by the vaporous medium to the tissue site is sufficient to cauterize tissue at the tissue site.

27. The apparatus of claim 23, wherein the amount of thermal energy transferred by the vaporous medium to the tissue site is sufficient to coagulate tissue at the tissue site.

28. The apparatus of claim 23, wherein the amount of thermal energy transferred by the vaporous medium to the tissue site is sufficient to shrink tissue at the tissue site.

29. The apparatus of claim 23, wherein the amount of thermal energy transferred by the vaporous medium to the tissue site is sufficient to seal tissue at the tissue site.

30. The apparatus of claim 19, further comprising a controller to control at least one of direction, duration, pressure, temperature, and volume of the vaporous medium.

31. The apparatus of claim 19, wherein the vaporous medium includes steam.

32. A system comprising:
a source that generates a vaporous medium; and
a surgical instrument that delivers the vaporous medium to a tissue site of a patient.

33. The system of claim 32, further comprising an insulated conduit that transports the vaporous medium from the source to the surgical instrument.

34. The system of claim 32, wherein the source that generates the vaporous medium is one of a radio frequency vapor generator and a resistive vapor heater.

35. The system of claim 32, wherein the source that generates the vaporous medium is external to the surgical instrument.

36. The system of claim 32, wherein the source that generates the vaporous medium is internal to the surgical instrument.

37. The system of claim 29, wherein the vaporous medium includes steam.

38. The system of claim 29, further comprising a controller to control at least one of direction, duration, pressure, volume, and temperature of the vaporous medium.

39. The system of claim 29, wherein the vaporous medium transfers an amount of thermal energy to the tissue site, the apparatus further comprising a controller to control the amount of thermal energy transferred by the vaporous medium.

40. The system of claim 39, wherein the amount of thermal energy transferred by the vaporous medium to the tissue site is sufficient to ablate tissue at the tissue site.

41. The system of claim 39, wherein the amount of thermal energy transferred by the vaporous medium to the tissue site is sufficient to provide hemostasis at the tissue site.

42. The system of claim 39, wherein the amount of thermal energy transferred by the vaporous medium to the tissue site is sufficient to cauterize tissue at the tissue site.

43. The system of claim 39, wherein the amount of thermal energy transferred by the vaporous medium to the tissue site is sufficient to coagulate tissue at the tissue site.

44. The system of claim 39, wherein the amount of thermal energy transferred by the vaporous medium to the tissue site is sufficient to shrink tissue at the tissue site.

45. The system of claim 39, wherein the amount of thermal energy transferred by the vaporous medium to the tissue site is sufficient to seal tissue at the tissue site.

46. The system of claim 32, wherein the surgical instrument is one of a catheter, scalpel, scissors, forceps, needle, and wand.

47. The system of claim 32, further comprising a monitor to observe the delivery of the vaporous medium to the tissue site.

48. The system of claim 47, wherein the vaporous medium includes at least one of salt, iodine, lidocaine, and chemotherapeutic materials.

49. A device comprising
an outer housing;
an inner liquid conduit that is in fluid communication with a liquid supply conduit that supplies liquid to the device;
a vapor outlet;
an inner vessel that defines a chamber adjacent the distal end, the chamber being in fluid communication with the inner liquid conduit via an inlet port and in fluid communication with the vapor outlet via an exit port;
electrical leads extending from the proximal end of the housing, one lead coupled to the inner vessel and one lead coupled to the inner liquid conduit; and
a radio frequency (RF) generator coupled to the proximal end of the electrical leads to apply a RF current to heat the fluid in the chamber and thereby create a vaporous medium, the vaporous medium being emitted from the chamber via the vapor outlet.

50. The device of claim 49, further comprising a liquid supply conduit to supply liquid from a liquid supply.

51. The device of claim 49, further comprising an electrically insulating sheath to match the impedance of the radio frequency generator.

52. The device of claim 49, further comprising a valve to prevent fluid flow in the reverse direction into the chamber via the vapor outlet.

53. The device of claim 49, wherein the outer housing is electrically insulated.

54. The device of claim 49, further comprising a controller to control at least one of direction, duration, volume, pressure, and temperature of the vaporous medium.

55. The device of claim 49, further comprising a controller to control a pump that supplies liquid to the surgical instrument.

56. The device of claim 49, further comprising a controller to control the RF generators.

57. The device of claim 49, further comprising a connector interface that couples the liquid supply conduit in fluid communication with the inner liquid conduit.

58. The device of claim 49, further comprising an insulated seal to enclose oppsing ends of the chamber.

59. A method comprsing delivering a vaporous medium to a tissue site of a patient, the vaporous medium having a temperature selected to cause at least one of ablation, hemostasis and tissue shrinkage within a portion of the tissue site.

60. A device comprising a housing to contain a vaporous medium, and a port to direct the vaporous medium at a tissue site of a patient.

61. A method comprising delivering a vaporous medium to a tissue site of a patient,
wherein a portion of the vaporous medium travels within interstitial spaces between tissue cells at the tissue site.
